(19) United States
(12) Patent Application Publication
(10) Pub. No.: US 2014/0037062 A1
(43) Pub. Date: Feb. 6, 2014

(54) IMAGE GUIDED RADIATION THERAPY

(71) Applicants: Daniel Robert ELGORT, New York, NY (US); Lucian Remus ALBU, Forest Hills, NY (US); Clemens BOS, Eindhoven (NL)

(72) Inventors: Daniel Robert ELGORT, New York, NY (US); Lucian Remus ALBU, Forest Hills, NY (US); Clemens BOS, Eindhoven (NL)

(73) Assignee: KONINKLIJKE PHILIPS ELECTRONICS N.V., EINDHOVEN (NL)

(21) Appl. No.: 13/563,788

(22) Filed: Aug. 1, 2012

(51) Int. Cl. A61B 6/08

(52) U.S. Cl. 378/63; 378/65

(57) ABSTRACT

An image guided radiation therapy system comprises a radiation source to generate radiation. Radiation optics forms a therapeutic radiation beam from the therapeutic radiation from the radiation source. An imaging system forms an image of a target zone to control the radiation optics to direct the therapeutic radiation beam onto the target zone. The radiation optics is provided with an optics module configured to generate an imaging photonic beam endowed with optical angular momentum. The imaging system comprises a magnetic resonance assembly to receive magnetic resonance signals the from the target zone generated by imaging photonic beam endowed with optical angular momentum.

### FIELD OF THE INVENTION

The invention pertains to an image guided radiation therapy system.

### BACKGROUND OF THE INVENTION

An image guided radiation therapy system is known from the international application WO2010/067227.

The known image guided radiation therapy system comprises a magnetic resonance imaging system for acquiring magnetic resonance imaging data in an imaging zone. A guiding means guides a beam of charged particles to a target zone within a subject. The imaging zone contains the target zone and the target zone within the subject is determined using the magnetic resonance imaging data. The beam of charged particles encloses an angle with the magnetic field lines less than 30°.

### SUMMARY OF THE INVENTION

An object of the invention is to provide an image guided radiation therapy system that more accurately controls the therapeutic radiation beam onto the target zone. This object is achieved by an image guided radiation therapy system according to the invention which comprises

a radiation source to generate radiation,

radiation optics to form a therapeutic radiation beam from the therapeutic radiation from the radiation source,

an imaging system to form an image of a target zone and to control the radiation optics to direct the therapeutic radiation beam onto the target zone, wherein

the radiation optics is provided with an optics module configured to generate an imaging photonic beam endowed with optical angular momentum and

the imaging system comprises a magnetic resonance assembly to receive magnetic resonance signals the from the target zone generated by imaging photonic beam endowed with optical angular momentum

The imaging photonic beam endowed with orbital angular momentum generates nuclear hyperpolarisation in the tissue of the subject, e.g. a patient to be examined, onto with the photonic beam is directed. Owing to the generated hyperpolarisation magnetic resonance signals are generated from the tissue onto which the OAM photonic beam is directed. The magnetic resonance imaging assembly then produces a magnetic resonance image of the tissue that is illuminated by the OAM photonic beam. This magnetic resonance image is reconstructed from the magnetic resonance signals generated by the OAM photonic beam. An insight of the present invention is that the magnetic resonance image generated on the basis of the OAM photonic beam can be used for image guiding the therapeutic radiation beam. Another insight of an example of the present invention is that as the both the therapeutic radiation beam and the OAM photonic beam are formed by the radiation optics, the beam paths of the therapeutic radiation beam and the OAM photonic beam are in a well defined mutual spatial relationship. On the basis of this spatial relationship between the therapeutic radiation beam and the OAM photonic beam, it is achieved to image the region in which the therapeutic beam is actually impinging on in the patient's anatomy. Thus, on the basis of the magnetic resonance image generated on the basis of the hyperpolarisation generated by the OAM photonic beam, the therapeutic radiation beam can be monitored and accurately directed onto the target zone. That is, the OAM photonic beam causes a hyperpolarised region of tissue within the patient to be examined which appears as a hyperintense region in the magnetic resonance image. This hyperintense region shows the position and orientation of the therapeutic radiation beam with respect to the patient to be examined. Notably, the target zone includes a lesion such as a tumour to be treated by the therapeutic radiation beam.

In further examples of the invention, the therapeutic radiation beam may be a high-energy x-ray beam, a γ-ray beam or the therapeutic radiation beam may be a particle beam such as a proton beam, a heavy-ion beam or a β-radiation beam.

In a preferred mode of operation of the image guided therapy system of the invention, the optics module generates the imaging OAM photonic beam shortly before the radiation source is activated. In this way the position and orientation of the therapeutic radiation beam path can be verified accurately. This verification does not deposit any significant radiation dose (e.g. x-ray dose) to the patient.

Further, the optics module includes an optical system for generating the OAM photonic beam with polarisers, beam expander (to enable the beam to fill a forked hologram), a diffractive grating with the forked hologram pattern, a spatial filter (to select the diffraction component with the OAM), and focusing lenses. To ensure the optical system works for high values of the optical angular momentum of the photonic beam (1-values, the size of the spatial filter and the aperture of the other optical elements will need to be increased in accordance with the radius of the photonic beam with OAM increasing with 1-value). As a relatively weak stationary magnetic field is needed only to establish the precession frequency of the hyperpolarised nuclei (i.e. hyperpolarised nuclear spin moments), only a simple magnet is sufficient which can be employed outside of the body of the patient to be examined. From the acquired magnetic resonance signals magnetic resonance spectral data are derived by the magnetic resonance spectrometer. The generation of the magnetic resonance signals from the OAM photonic beam is known per se from the international application WO 2009/081360-A1.

These and other aspects of the invention will be further elaborated with reference to the embodiments defined in the dependent Claims.

In one aspect of the invention, the optics module is configured to form both the therapeutic radiation beam and the imaging photonic beam endowed with optical angular momentum from the radiation source. This achieves that the beam paths of the imaging photonic beam and of the therapeutic radiation beam are easily in a well defined mutual spatial relationship. Moreover, the radiation source is efficiently used in that it is the basis of both the OAM photonic beam and the therapeutic radiation beam. In one further aspect of the invention the therapeutic radiation beam itself may be endowed with optical angular momentum and serve as the OAM photonic beam.

A next aspect of the invention, the optics module is provided with a separate imaging radiation source, for example an x-ray source. This aspect of the invention achieves that different types of radiation can be employed for the therapeutic radiation beam and the imaging photonic beam, respectively. This allows to employ different types of radiation for the therapeutic radiation beam and the imaging photonic beam. For example the therapeutic radiation beam may be a particle beam and the imaging photonic beam may be an x-ray beam. Thus, on the one hand the electromagnetic radiation for OAM photonic beam can be selected to yield good imaging results to form a high quality rendition of the target zone. On the other hand, the therapeutic radiation can be selected to achieve optimum therapeutic effect upon irradiation of the target zone.

In a further aspect of the invention, the imaging OAM photonic beam has a range of energies. This range of energies causes hyperpolarisation in an elongate region in the tissue. This is due to higher energies having a larger penetration depth in the tissue, the hyperpolarisation is generated by the OAM photonic beam over a wider range along its propagation direction, while in the direction transverse to the propagation direction the hyperpolarisation is generated over a uniform range, at least having a range that is narrower as compared to the range along the propagation direction. Notably, a more or less cylindrically shaped or ellipsoid shaped hyperpolarisation range is formed. Then, the beam path of the OAM photonic beam is along the long axis of the elongate region, for example of the ellipsoid and the therapeutic radiation beam can be accurately aligned with the thus visualised beam path. This allows easy and accurate visualisation of the area covered by the therapeutic radiation beam. Also the position and orientation of the therapeutic radiation beam are well visualised by the hyper intense region in the magnetic resonance image. For example, the therapeutic radiation beam path may be adjusted to be along the long axis of the elongate region in the tissue where hyperpolarisation is generated by the imaging OAM photonic beam.

In a further aspect of the invention, the therapeutic radiation beam has an energy range of for example 6-10 MeV which has a good efficacy in treating a tumour in that necrosis is created in the region where the therapeutic radiation beam is absorbed by the (cancerous) tissue. The imaging OAM photonic beam has for example an energy range on 10-100 keV, which has a good penetration depth in tissue to reach a lesion or tumour in the patient's anatomy.

In another aspect of the invention, the optics module is configured to generate multiple imaging OAM photonic beams. Notably, these imaging OAM photonic beams are directed to different locations around the beam path of the therapeutic radiation beam. This aspect of the invention achieves a good delimitation in the magnetic resonance image of the region covered by the therapeutic radiation beam. Notably, the hyper intense regions generated by the respective imaging OAM photonic beams provided a good visualisation of the delimitation of the boundaries of the therapeutic radiation beam in the patient's anatomy. Thus, it is achieved to accurately control the radiation optics to direct the therapeutic radiation beam onto the target zone to impinge onto the lesion or the tumour, and avoid radiation damage to sensitive healthy tissue in the neighbourhood of the target zone.

These and other aspects of the invention will be elucidated with reference to the embodiments described hereinafter and with reference to the accompanying drawing wherein

### BRIEF DESCRIPTION OF THE DRAWINGS

FIG. 1 shows a schematic representation of an image guided radiation therapy system in which the present invention is implemented;

FIG. 2 shows a schematic representation of details of the optical module and

FIG. 3 shows a schematic diagram of the image guided radiation therapy system of the invention in the form of a hybrid LINAC-MRI system.

### DETAILED DESCRIPTION OF THE EMBODIMENTS

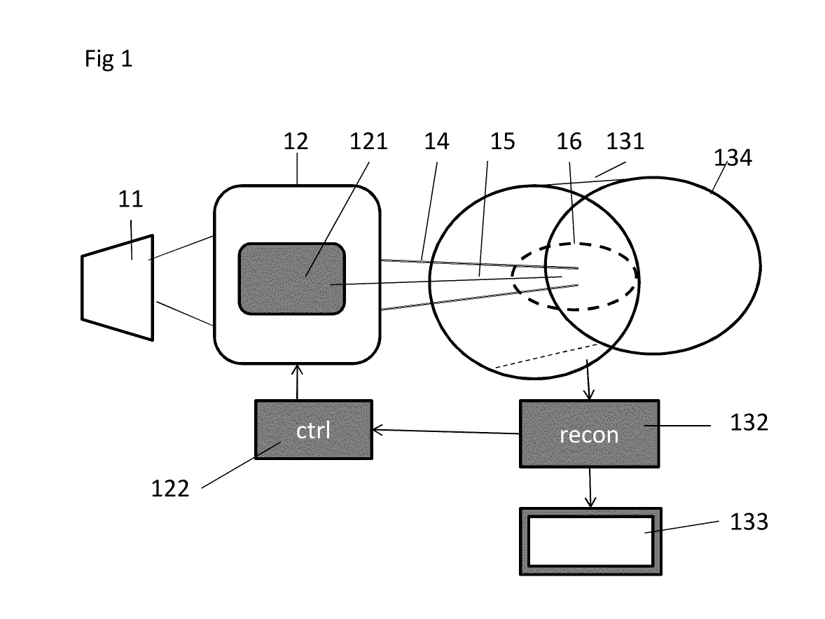

FIG. 1 shows a schematic representation of an image guided radiation therapy system in which the present invention is implemented. The radiation source 11 emits therapeutic radiation into the radiation optics 12 which forms the therapeutic beam 14. The therapeutic radiation may be high-energy electromagnetic radiation such as γ-radiation, hard x-radiation. The radiation optics is configured to target toe therapeutic beam onto the target zone 16 e.g. in a patient to be treated. In particular the radiation source and radiation optics may be formed as a linear accelerator (LINAC) system which produces a high energy electron beam that is aimed onto an anode target so as to generate emission of high-energy electromagnetic radiation from the anode target. In the example of the LINAC, the radiation source 11 includes a cathode from which a high-energy electron beam is emitted is formed as for emitting the electron beam onto an anode. An electro-magnetic lens system is provided in the radiation source 11 to direct the electron beam onto the anode. The impinging high-energy electrons cause the material of the anode to emit high-energy x-radiation that forms the therapeutic radiation in this example. The radiation optics 12 includes a beam collimator, preferably in the form of a multi leaf collimator to form the high-energy radiation beam 14 from the high-energy radiation that is emitted from the anode.

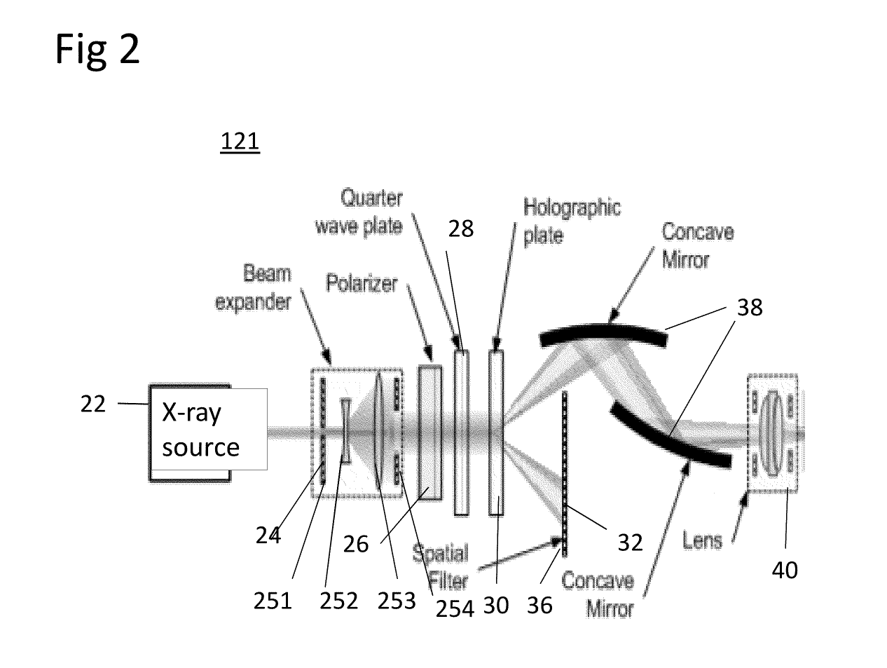

An imaging system 13 is provided to monitor the correct aim of the high-energy radiation beam 14 onto the target zone 16. The imaging system 13 includes the magnetic resonance assembly 131 to produce magnetic resonance images of the object to be irradiated. The magnetic resonance signals in the object, notably in the target zone are generated by the OAM photonic radiation beam 15 that is generated by the optics module 121 that is included in or mounted onto the radiation optics 12. Alternatively, the optics module may be mounted opposite the radiation source on a common gantry 137. In this way the beam path of the high-energy radiation beam and of the OAM photonic beam 15 can be co-registered in that there is a fixed and stable geometrical relationship between both beam paths. Details of the optics module are shown in FIG. 2. The OAM photonic beam 15 is endowed with optical angular momentum which may interact the with material (tissue) of the target zone 16 and create (nuclear) hyperpolarisation in the target zone 16. From the magnetic resonance signals acquired from the target zone an magnetic resonance image is reconstructed by the reconstructor 132, for example by way of a fast-Fourier transform method. The magnetic resonance image is displayed on a monitor 133. The magnetic resonance assembly includes a main magnet 134 for generating a stationary baseline magnetic field to define a Larmor precession frequency of the hyperpolarised nuclei, gradient coils 135 to generate gradient magnetic fields for spatial (frequency and phase encoding) encoding of the magnetic resonance signals and an RF coil for acquiring the magnetic resonance signals. Notably, the gradient coils 135 are implemented as split gradient coils having two gradient coils sections that are separated by an opening that allows the high-energy beam 14 as well as the OAM photonic beam 15 to pass to the target zone 16. The OAM photonic beam 15 is co-registered with the high-energy beam 14 so that the region where hyperpolarisation is generated by the optical angular momentum corresponds with the region where the high-energy radiation beam is absorbed by the material, i.e. tissue. The hyperpolarised region shows up as a hyperintense region in the magnetic resonance image. This enables easy and accurate adjustment of the beam path of the high-energy radiation to coincide with target zone 16 that is to be irradiated. The adjustment of the beam path of the high-energy radiation beam is carried-out by way of a control unit 122 that receives image information of the magnetic resonance image from the reconstructor. From the orientation and position for the hyperintense region and a treatment plan that represents the target zone 16 to be irradiated, the control unit has the function to calculate the required beam path and control the radiation source to direct the high-energy radiation beam along the calculated beam path onto the target zone.

FIG. 2 shows a schematic representation of details of the optical module. In FIG. 2, an exemplary arrangement of optical elements is shown for endowing light with OAM. It is to be understood that any electromagnetic radiation can be endowed with OAM, not necessarily only visible light. The described embodiment uses soft x-rays, which interacts with the molecules of interest, and has no damaging effect on living tissue. Light/radiation above or below the visible spectrum, however, is also contemplated. An x-ray source 22 produces x-radiation that is sent to a beam expander 24. Preferably, the x-radiation has an energy n the range of 10-100 keV which provides for an adequate penetration depth into the tissue of the patient to be treated. The beam expander 24 includes an entrance collimator 251 for collimating the emitted light into a narrow beam, a concave or dispersing lens 252, a refocusing lens 253, and an exit collimator 254 through which the least dispersed frequencies of light are emitted. In one embodiment, the exit collimator 254 narrows the beam to a 1 mm beam.

After the beam expander 24, the light beam is circularly polarized by a linear polarizer 26 followed by a quarter wave plate 28. The linear polarizer 26 takes unpolarised light and gives it a single linear polarization. The quarter wave plate 28 shifts the phase of the linearly polarized light by ¼ wavelength, circularly polarizing it. Using circularly polarized light is not essential, but it has the added advantage of polarizing electrons.

Next, the circularly polarized light is passed through a phase hologram 30. The phase hologram 30 imparts OAM and spin to an incident beam. The value “1” of the OAM is a parameter dependent on the phase hologram 30. In one embodiment, an OAM value 1=40 is imparted to the incident light, although higher values of 1 are theoretically possible. The phase hologram 30 is a computer generated element and is physically embodied in a spatial light modulator, such as a liquid crystal on silicon (LCoS) panel, 1280×720 pixels, 20×20 μm2, with a 1 μm cell gap. Alternately, the phase hologram 30 could be embodied in other optics, such as combinations of cylindrical lenses or wave plates. The spatial light modulator has the added advantage of being changeable, even during a scan, with a simple command to the LCoS panel.

Not all of the light that passes through the holographic plate 30 is imparted with OAM and spin. Generally, when electromagnetic waves with the same phase pass through an aperture, it is diffracted and projected into a pattern of concentric circles some distance away from the aperture (Airy pattern). The bright spot (Airy disk) in the middle represents the 0th order diffraction, in this case, that is light with no OAM. Circles adjacent the bright spot represent diffracted beams of different harmonics that carry OAM. This distribution results because the probability of OAM interaction with molecules falls to zero at points far from the centre of the light beam or in the centre of the light beam. The greatest chance for interaction occurs on a radius corresponding to the maximum field distribution, that is, for circles close to the Airy disk. Therefore, the maximum probability of OAM interaction is obtained with a light beam with a radius as close as possible to the Airy disk radius.

A spatial filter 36 is placed after the holographic plate to selectively pass only light with OAM and spin. The 0th order spot 32 always appears in a predictable spot, and thus can be blocked. As shown, the filter 36 allows light with OAM to pass. Note that the filter 36 also blocks the circles that occur below and to the right of the bright spot 32. Since OAM of the system is conserved, this light has OAM that is equal and opposite to the OAM of the light that the filter 36 allows to pass. It would be counterproductive to let all of the light pass, because the net OAM transferred to the target molecule would be zero. Thus, the filter 36 only allows light having OAM of one polarity to pass. The diffracted beams carrying OAM are collected using concave mirrors 38 and focused to the region of interest with a fast microscope objective lens 40. The mirrors 38 may not be necessary if coherent light were being used. A faster lens (having a high f-number) is desirable to satisfy the condition of a beam waist as close as possible to the size of the Airy disk. In alternate embodiments, the lens 40 may be replaced or supplemented with an alternative light guide or fibre optics.

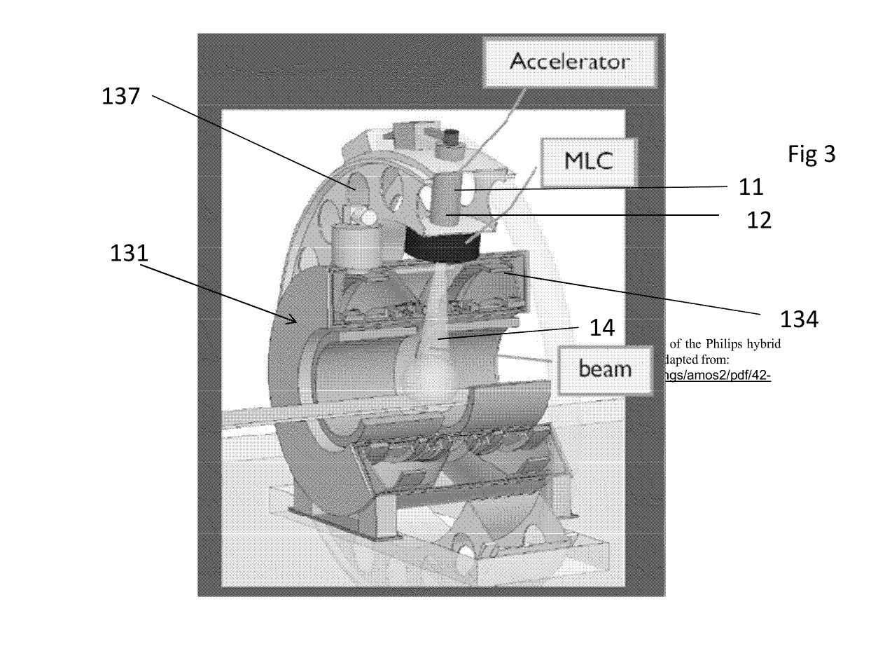

FIG. 3 shows a schematic diagram of the image guided radiation therapy system of the invention in the form of a hybrid LINAC-MRI system. The radiation source 11 with the accelerator to generate the electron beam is mounted on a gantry 137. The therapeutic radiation beam 14 in this example is the high-energy x-ray beam that is emitted from the anode in the radiation source onto which the electron beam impinges. The beam path of the therapeutic radiation beam 14 passes in between the magnetic resonance examination assembly's main magnet coils 134. The cross section of the therapeutic radiation beam is shaped by the multi-leaf collimator (MLC) that is incorporated in the radiation source 11. The optics module 12 is also incorporated in the radiation source and provides the OAM photonic beam that is guided along its beam path. The optics module is mounted in the radiation source in such a way that the beam paths of the OAM photonic beam and the therapeutic radiation beam have essentially the same central longitudinal axes. In this way it is ensured that the OAM photonic beam generates (nuclear) hyperpolarisation in the region into which the radiation source deposits its high-energy radiation.

1. An image guided radiation therapy system comprising
a radiation source to generate radiation,
radiation optics to form a therapeutic radiation beam from the therapeutic radiation from the radiation source,
an imaging system to form an image of a target zone and to control the radiation optics to direct the therapeutic radiation beam onto the target zone, wherein
the imaging system includes an optics module configured to generate an imaging photonic beam endowed with optical angular momentum and
the imaging system comprises a magnetic resonance assembly to receive magnetic resonance signals the from the target zone generated by imaging photonic beam endowed with optical angular momentum.

2. An image guided radiation therapy system as claimed in claim 1, is provided with wherein the optics module configured to generate an imaging photonic beam endowed with optical angular momentum in mounted in the radiation optics

3. An image guided radiation therapy system as claimed in claim 1, wherein the optics module is configured to form the imaging photonic beam endowed with optical angular momentum from the radiation source.

4. An image guided radiation therapy system as claimed in claim 1, wherein the optics module is provided with an imaging radiation source, notably an x-ray source.

5. An image guided radiation therapy system as claimed in claim 1, wherein the imaging photonic beam endowed with optical angular momentum has a range of energies.

6. An image guided radiation therapy system as claimed in claim 1, wherein the imaging photonic beam endowed with optical angular momentum has an energy range of 10-100 keV and the therapeutic radiation beam has an energy range of 6-10 MeV.

7. An image guided radiation therapy system as claimed in claim 1, wherein the optics module is configured to produce multiple, e.g. four, imaging photonic beams endowed with optical angular momentum, directed at different locations around the therapeutic radiation beam path.
